(19) United States
(12) Patent Application Publication
(10) Pub. No.: US 2020/0129221 A1
(43) Pub. Date: Apr. 30, 2020

(54) KIT AND DOSAGE REGIMEN FOR NON-SURGICAL TREATMENT OF SKIN LESIONS

(71) Applicant: VERSAILLES B.V., Diemen (NL)

(72) Inventors: An De Naeyer, Gentbrugge (BE); Mark Berkhout, Diemen (NL)

(21) Appl. No.: 16/606,753

(22) PCT Filed: Apr. 17, 2018
(86) PCT No.: PCT/EP2018/059767

§ 371 Date: Oct. 20, 2019

(30) Foreign Application Priority Data
Apr. 20, 2017 (NL) 2018751

(51) Int. Cl. A61B 18/02; A61K 9/00

(52) U.S. Cl. CPC A61B 18/0218; A61K 31/60; A61K 9/0014

(57) ABSTRACT

The current invention concerns a kit suitable for the non-surgical treatment of skin lesions including warts, verrucae, corns, and skin tags said kit comprising a cryogenic body comprising a housing hosting a coolant container and further comprising a cryogenic applicator head, whereby the housing and the cryogenic applicator head extend axially; said cryogenic applicator head is provided with a cryogenic tip for performing a cryogenic treatment to a skin lesion, whereby said coolant container contains a coolant suitable for cooling said tip; the kit further comprises an acid body comprising an acid container containing an acidic composition and having an acid outlet, and an acid dispenser head that axially extends from said acid container, is in fluid communication with said acid outlet, and is suitable for applying a dose of said acidic composition to a skin lesion. The invention also concerns a dosage regimen.

### TECHNICAL FIELD

The invention pertains to the technical field of the non-surgical treatment of skin lesions including warts, verrucae, corns, and skin tags.

### BACKGROUND OF THE INVENTION

Typical wart treatments are topical treatments. One particularly popular treatment involves the application of an acidic composition to the wart on a regular basis, the so-called acid treatment. Another popular method of treatment is the so-called cryogenic treatment.

The acidic compositions used as topical treatments for warts are mostly keratolytic. This means that they break down keratin and thereby desquamate the tissue of the wart. Most of the compounds are irritants and some are directly necrotic. The most commonly used compositions are those containing salicylic acid. Typically, the composition is applied to the wart on a daily basis. The treated areas may be kept covered with a bandage or other occlusive material. Desquamation and peeling of the tissue occur, and eventually the wart is reduced in size and may ultimately regress completely. The treatment generally takes a long time, up to several weeks. Salicylic acid has the advantages of being available without a prescription, being applicable at home, and having relatively low toxicity. Disadvantages are that it can damage healthy skin, that it is often not entirely effective so that recurrences are frequent, and that secondary infections may arise in the treatment-damaged skin.

Cryogenic treatments entail topically freezing a wart. These treatments typically include the use of a coolant liquid that extracts heat from its environment upon evaporation (so-called evaporative cooling). In order to remove the wart, it is important that the infected tissue region is frozen as a whole. This is conveniently accomplished by the direct or indirect application of the coolant, usually liquid nitrogen. Liquid nitrogen is effective but painful due to the extremely low temperature involved. Additionally, there are practical issues involved in the storage and handling of liquid nitrogen. This renders difficult the design of devices suitable for liquid nitrogen-based self-treatment, applied by the patients themselves at home. Generally speaking, such devices are therefore relatively expensive. In this respect, cryotherapy self-treatment devices employing coolants other than liquid nitrogen, such as disclosed in U.S. Pat. No. 6,387,090, stand as a valuable alternative. The coolant mostly employed is a mixture of dimethyl ether and propane (DMEP). Several types of cryogenic self-treatment applicators exist. In the most basic composition, the applicator is suitable for spraying the coolant liquid directly to the wart tissue. Slightly more advanced applicators include a porous tip, for instance a foam tip, to which the coolant liquid is supplied and which makes contact with the wart tissue. EP 2 759 272 discloses an even more advanced design of the cryogenic applicator, which now comprises a closed contact tip, for instance a metal tip, in contact with porous heat exchanger. The coolant liquid is now supplied to the heat exchanger instead, and does not contact the wart tissue directly. It is mentioned that the cooling temperature can be better controlled this way, through the specific design of the applicator and through the amount of cooling liquid that is supplied to the heat exchanger. Such devices are useful for self-treatment, although they typically cannot achieve the intensely low temperatures of liquid nitrogen. Hence, these are less effective in freezing the root portion of a deeply embedded wart. The wart may thus re-grow, even after repeated treatment. Such treatments can also be painful, although typically less so than liquid nitrogen.

Hence, there is a need in the art for a device/kit that is suitable for performing self-treatment of warts. Said device/kit should be safe and easy-to-use. Said device/kit should additionally have a relatively simple design, such that manufacturing costs can be reduced. Also, the device/kit should be such that the treatment method is less painful yet more effective than the treatments currently known, preferably involving less treatment sessions. There is thus also a need in the art for a dosage regimen related to said treatment. The present invention aims to resolve at least some of the problems mentioned above.

### SUMMARY OF THE INVENTION

In a first aspect, the invention provides a kit according to claim 1, suitable for the non-surgical treatment of skin lesions including warts, verrucae, corns, and skin tags, said kit comprising:
a cryogenic body comprising a housing hosting a coolant container and further comprising a cryogenic applicator head, whereby the housing and the cryogenic applicator head extend axially; said cryogenic applicator head comprises an applicator end portion and is provided at said applicator end portion with a cryogenic tip for performing a cryogenic treatment to a skin lesion, whereby said coolant container contains a coolant suitable for cooling said tip, and
an acid body comprising an acid container containing an acidic composition and having an acid outlet, and further comprising an acid dispenser head that axially extends from said acid container; said acid dispenser head is in fluid communication with said acid outlet and is suitable for applying a dose of said acidic composition to a skin lesion.

An advantage of the kit according to the present invention is that said kit makes it possible to perform both an acid treatment and a cryogenic treatment to a skin lesion. The kit thereby conveniently provides all objects that are necessary in performing each of both treatments.

In a second aspect, the invention provides a dosage regimen according to claim 15, suitable for the non-surgical treatment of skin lesions including warts, verrucae, corns, and skin tags; in particular, the dosage regimen is employed for treating a skin lesion, thereby comprising:
performing a cryogenic treatment to a skin lesion, and
applying 5 microliter up to 40 microliter of an acidic composition to said skin lesion, twice a day during two to eight days; said acidic composition comprises salicylic acid, formic acid, lactic acid, trichloroacetic acid, monochloroacetic acid, one or more fruit acids, and/or any combination thereof.

An advantage of performing an acid treatment prior to a cryogenic treatment is that the acid treatment has, among other things, a keratolytic action. The acid treatment thereby weakens the tissue of the skin lesion. This makes it easier for the cryogenic treatment to act on the wart, thereby improving the efficacy of the cryogenic treatment.

An advantage of performing a cryogenic treatment prior to an acid treatment is that the cryogenic treatment has a necrotic effect. The cryogenic treatment thereby weakens the tissue of the skin lesion, such that the acidic composition can penetrate faster and/or deeper into the wart tissue. This improves the efficacy of the acid treatment.

Another advantage is that the combination of acid treatments and cryogenic treatments has a synergistic effect. It should be stressed that the cryogenic treatment and the acid treatment is not merely a pre-treatment and a treatment, in any order. The cryogenic treatment is a treatment on itself, and the acid treatment is a treatment on itself, since it includes at least one of the acids mentioned above. The combination of these two, separate treatments has been found to show a synergistic effect.

### DESCRIPTION OF FIGURES

FIGS. 1a-f schematically depict a non-exhaustive collection of possible embodiments of the kit according to the present invention.

FIG. 2 shows an axial cut of the acid body, according to an embodiment of the invention.

FIGS. 3a and 3b give a perspective view on the applicator end portion of the cryogenic applicator head according to an embodiment of the invention, respectively without and with a cryogenic tip.

FIG. 4 shows an axial cut of the kit in its engaged configuration, according to an embodiment of the invention.

FIG. 5a gives a perspective view on the kit in its engaged configuration, according to an embodiment of the invention.

FIG. 5b gives a perspective view on the kit, during steps prior to an acid treatment.

FIG. 5c gives a perspective view on the kit, during steps prior to a cryogenic treatment.

### DETAILED DESCRIPTION OF THE INVENTION

The present invention concerns a kit for the non-surgical treatment of skin lesions including warts, verrucae, corns, and skin tags. It is an object of the invention to provide for a kit for performing a self-treatment of such skin lesions, whereby the kit is safe and easy-to-use, and has a simple design. Also, the kit should be such that the associated treatment is less painful yet more effective than the treatments currently known, preferably involving less treatment sessions. Moreover, it is the purpose of the present invention to provide for a dosage regimen for the non-surgical treatment of skin lesions including warts, verrucae, corns, and skin tags, whereby said dosage regimen is related to said treatment.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight” (weight percent), here and throughout the description unless otherwise defined, refers to the relative weight of the respective component based on the overall weight of the formulation.

In a first aspect, the invention provides a kit suitable for the non-surgical treatment of skin lesions including warts, verrucae, corns, and skin tags, said kit comprising:
a cryogenic body comprising a housing hosting a coolant container and further comprising a cryogenic applicator head, whereby the housing and the cryogenic applicator head extend axially; said cryogenic applicator head comprises an applicator end portion and is provided at said applicator end portion with a cryogenic tip for performing a cryogenic treatment to a skin lesion, whereby said coolant container contains a coolant suitable for cooling said tip, and
an acid body comprising an acid container containing an acidic composition and having an acid outlet, and further comprising an acid dispenser head that axially extends from said acid container; said acid dispenser head is in fluid communication with said acid outlet and is suitable for applying a dose of said acidic composition to a skin lesion.

The term “cryogenic body” covers at least those parts of the kit that are essential in performing the cryogenic treatment. The kit is at least configured to perform such a treatment to a skin lesion. Thereby, the “cryogenic body” does not comprise parts that are comprised by the “acid body”. The coolant container contains a coolant that advantageously enables evaporative cooling of the tissue of the skin lesion. To this end, the cryogenic tip is cooled using the coolant and simultaneously and/or subsequently brought in contact with said tissue. Preferably, the coolant is contained under pressure in a suitable coolant container; it will evaporate and thus extract heat from its environment, e.g. from the applicator tip, upon release from said container. A standard type of gas cartridge is a suitable container, although the man skilled in the art is able to choose alternative coolant containers. The cryogenic body also comprises a housing hosting said gas cartridge. Preferably said housing is adapted for hosting a standard type of gas cartridge on one hand, and for providing an easy-to-grab outer side on the other hand. At least said housing has an easy-to-grab outer side. This way, it is advantageously possible to firmly grab the housing using one hand. Preferably, the cryogenic body is an elongated body, whereby the axial direction is defined in relation to said body, along the length of it.

The term “acid body” covers at least those parts of the kit that are essential in performing the acid treatment. The kit is at least configured to perform such a treatment to a skin lesion. Thereby, the “acid body” does not comprise parts that are comprised by the “cryogenic body”. The acid body comprises an acid container containing the acidic composition. Preferably, the material of the inner walls of said container is such that it does not rapidly degrade in an acidic environment. Even more preferably, the acid container is made of polypropylene, yet any other suitable material can be selected by the man skilled in the art. Optionally, the inner walls of the acid container are provided with a surface coating that provides for protection against acidic environments. Preferably, the acid body is an elongated body, whereby the axial direction is defined in relation to said body, along the length of it. In a preferred embodiment, the acid outlet of the acid container is provided with a valve system. The design of the acid body and, more specifically, said valve system is thereby such that the release of the acidic composition to the acid dispenser head can be controlled externally. In a preferred embodiment, the acid container is internally provided with a piston for building up pressure within the acid container, upon which the acidic composition is released. Preferably such a piston can be operated externally. Yet, the acid container could alternatively be any suitable container known in the state of the art, such as a tube that can be compressed sideways.

An advantage of the kit according to the present invention is that said kit makes it possible to perform both an acid treatment and a cryogenic treatment to a skin lesion. The kit thereby conveniently provides all objects that are necessary in performing each of both treatments.

In a further preferred embodiment of the kit, the cryogenic body and the acid body are detachably engaged, thereby forming an elongated device.

The term “detachably engaged” (Dutch: “losmaakbaar verbonden”) means that both parts involved are engaged with each other in a non-permanent fashion. Preferably but not limited thereto, the first part has an end portion that defines a receptacle, whereby said receptacle is adapted to receive an end portion of the second part. In any case, said end portions are configured to co-act. Additionally, the detachably engaged parts can be separated. This separation does not involve the exertion of elevated forces on the parts. Nor does this separation involve deforming or altering the parts in a permanent way. Preferably, in the detachably engaged configuration, the acidic composition, the coolant and/or the cooled parts are shielded from the user of the kit. For releasing coolant or acidic composition, at least two detachably engaged parts should be separated from each other. In a further preferred embodiment of the detachably engaged parts, the parts are designed in such a way that they can be separated upon the execution of a specific scheme of sliding and/or turning motions of one part relative to the other. More generally it is preferred that a child cannot separate the detachably engaged parts: the detachable engagement should be child-locked. An advantage is that the kit is then safer for a child, because said child cannot separate the parts, whereby it could be subjected to the acidic composition, to the coolant and/or to cooled parts. In one embodiment, an advanced scheme of relative sliding and/or turning motions is required for separating two detachably engaged parts. A child will then not be able to execute this scheme. According to another embodiment, the detachably engaged parts extend in the axial direction, and the first part can only be separated from the second part when the relative azimuthal angle about the axis takes an exact value out of one or more specific, discrete values; preferably only when it takes one specific, discrete value. The probability for the child to separate the parts will then be extremely low.

An advantage is that the two separate parts can be brought in an engaged configuration (of the kit). In this engaged configuration, the kit advantageously comprises one larger part instead of two separate, smaller parts. At the same time, this engagement is not permanent, as stated above.

Preferably but not limited thereto, the kit as described throughout this document takes the engaged configuration most of the time. That is, when sold and during storage, but not necessarily when employed in performing a treatment. It is therefore equally possible to interpret the kit as a device, whereby said device is suitable for the non-surgical treatment of skin lesions including warts, verrucae, corns, and skin tags.

According to an alternative embodiment of the kit, the cryogenic body is a first part of the kit and the acid body is a second part of the kit. Both parts are well-separated from each other, in a sense that it is not the case that both bodies are connected or are configured for being detachably engaged to form one integral part. Preferably, the cryogenic body then additionally comprises a removable cap for covering and protecting the acid dispensing head, while the acid body then preferably additionally comprises a removable cap for covering and protecting the cryogenic applicator head.

In a further or alternative, preferred embodiment of the kit, the housing and the cryogenic applicator head are detachably engaged, thereby forming an engaged configuration.

An advantage of this embodiment of the kit, whereby the cryogenic applicator head and the housing hosting the coolant container are detachably engaged, is that the two separate parts can be brought in an engaged configuration (of the cryogenic body). In this engaged configuration, the kit advantageously comprises one larger part instead of two separate, smaller parts. At the same time, this engagement is non-permanent, as stated above. Preferably, the coolant and/or the cooled parts are shielded from the user, in the engaged configuration of the housing and the cryogenic applicator head.

Another advantage is that both parts can be separated in a non-permanent way. For instance, the kit is suitable for being used in a cryogenic treatment. In a non-limiting further embodiment, cooling of the cryogenic tip is only possible in a detachably engaged configuration. The (cooled) cryogenic tip is thereby shielded from the user of said kit. Afterwards, the cryogenic applicator head is separated from the housing, such that it is exposed. It can then be used for applying a cryogenic treatment to a skin lesion. The cryogenic applicator head is thereby either handled directly or indirectly, via an easy-to-grab member to which is connected or with which it is detachably engaged. In such case, all body contact with the housing hosting the coolant container is avoided as much as possible. This is advantageous, because the coolant container contains a coolant under pressure and might therefore be dangerous. Additionally, it is avoided to approach the coolant container itself to the skin lesion to be treated.

Preferably, the housing hosting the coolant container and the visible sides of the cryogenic applicator head are colored using substantially different colors. In a further or alternative, non-limiting embodiment, the visible side of said housing and/or of said cryogenic applicator head is provided with icons that give indications on how to separate these parts. This advantageously makes it easier for an adult to separate the cryogenic applicator head from the housing when detachably engaged. On the other hand, it does not actually help a young child in doing the same thing. It is thus child-safe.

In a further preferred embodiment of the kit, the acid body and the cryogenic body, in its engaged configuration, are detachably engaged, thereby forming an engaged configuration of the kit.

Preferably, the colors present on the visible sides of the acid body are substantially different from the colors present on the visible sides of the cryogenic body. In a further or alternative, non-limiting embodiment, the visible side of the acid body and/or of the cryogenic body is provided with icons that give indications on how to separate these parts. This advantageously makes it easier for an adult to separate the acid body from the cryogenic body when detachably engaged. On the other hand, it does not actually help a young child in doing the same thing. It is thus child-safe.

In a further preferred embodiment of the kit, the cryogenic applicator head is positioned between the coolant container and the acid body. Preferably, the cryogenic applicator head is detachably engaged with the acid body on one hand, and the coolant container or the housing hosting said container on the other hand.

An advantage is that, in the engaged configuration of the kit, the cryogenic applicator head is better protected; indeed, the cryogenic applicator head is covered at both sides.

In a further preferred embodiment of the kit, the cryogenic applicator head comprises a distal end portion, opposite to its applicator end portion, whereby said distal end portion is configured for receiving the acid dispenser head. Said distal end portion and said acid dispenser head are thereby configured for being detachably engaged, in a child-safe fashion.

An advantage is that the cryogenic applicator head now serves as a cover for the acid dispenser head. It is not necessary for the kit to comprise an additional, removable cap. Such removable caps are typically small parts, and therefore they easily get lost. Preferably, the outer wall of the acid container has an easy-to-grab design. Alternatively, the acid container is comprised by or hosted by an easy-to-grab holder. As stated above, the acid dispenser head axially extends from the acid container. Thus in both cases, the acid dispensing head can be conveniently handled indirectly using one hand, via said container or via said holder respectively. Another advantage is that the cryogenic applicator head can now conveniently be handled using one hand, via the same container or holder. Indeed, the cryogenic applicator head is configured for detachably engaging the acid dispensing head, whereby its applicator side portion axially extends from the acid body. Said applicator side portion, to which the cryogenic tip is provided, is thereby pointing in the same direction as the acid dispensing head. As the same easy-to-grab member is employed for handling both the acid dispenser head and the cryogenic applicator head, the kit can be made shorter, in engaged configuration.

In a further preferred embodiment of the kit, the housing comprises an end portion, whereby said end portion defines a cryogenic receptacle that is configured for receiving the applicator end portion of the cryogenic applicator head. Said cryogenic receptacle and said applicator end portion are thereby configured for being detachably engaged, in a child-safe fashion.

An advantage is that the cryogenic receptacle now serves as a cover for the applicator side of the cryogenic applicator head. It is not necessary for the kit to comprise an additional, removable cap.

In a further preferred embodiment of the kit, the coolant container has an outlet for the coolant and a valve communicating with said outlet, whereby the cryogenic receptacle is in coolant communication with said outlet. At the same time, the cryogenic receptacle is configured for being detachably engaged with the applicator end portion of the cryogenic applicator. As stated above, the cryogenic applicator head is provided at its applicator end portion with a cryogenic tip. Preferably, at least two specific schemes of relative sliding and/or turning motions of the cryogenic applicator head relative to the housing exist. At least one and more preferably just one of said schemes detachably engages the applicator side of the cryogenic applicator in the cryogenic receptacle. At least one other and more preferably just one other of said schemes opens the valve of the coolant container. Coolant is thereby supplied to the cryogenic receptacle, and therefore also to the applicator side of the cryogenic applicator head, thereby directly or indirectly cooling the cryogenic tip. Preferably, each of said schemes is child-safe.

An advantage of the latter configuration is that the cryogenic tip can be cooled while being received by the cryogenic receptacle. Said cryogenic receptacle additionally serves as a cover for the applicator side of the cryogenic applicator head, and thus also as a cover for the cryogenic tip. This is thus a safe configuration.

According to a first, further preferred embodiment of the kit, the cryogenic applicator head comprises a heat exchanger that is in heat exchange contact with the cryogenic tip, said cryogenic tip comprising a metal and/or a ceramic; in the engaged configuration of the cryogenic body, said heat exchanger is in coolant communication with the cryogenic receptacle; said heat exchanger further comprises a porous member adapted for conducting the coolant. For instance, the cryogenic tip is a stainless steel tip, an aluminum tip, or a brass tip. Preferably, said cryogenic tip at least comprises a ceramic.

The term “engaged configuration of the cryogenic body” thereby denotes that the housing comprising the coolant container is detachably engaged with the cryogenic applicator head as described above. In the engaged configuration of the cryogenic body, the heat exchanger receives coolant when the valve of the coolant container is opened. Thereafter, the coolant cools the heat exchanger and the cryogenic tip via evaporative cooling. In the current embodiment, it is preferred that the design of the cryogenic applicator head it avoids direct contact between the coolant and the skin lesion, during the cryogenic treatment. The skin lesion is thereby not “directly cooled”, but rather “indirectly cooled” via the tip, meaning that the coolant cools the tip, and the tip cools the skin lesion.

An advantage of indirectly cooling the skin lesion is that it is less painful. Another advantage is that the location of the cooling action can be better controlled, such that the risk of freezing healthy tissue is reduced. Another advantage is that the risk of over-freezing the tissue of the skin lesion is reduced.

An advantage of incorporating a porous member in the heat exchanger is that the porous member conducts and absorbs the coolant, while slowing down the coolant evaporation. The coolant therefore evaporates more gradually, such that low temperatures can be maintained for a longer period of time. Additionally, the porous member holds the coolant in the direct vicinity of the cryogenic tip, in heat exchange contact with the cryogenic tip.

Preferably, the applicator side of the cryogenic applicator head is provided with one or more openings towards the porous member of the heat exchanger. This way, evaporative cooling is enhanced such that increased cooling action with lower temperatures is obtained. Another advantage is that coolant can be supplied from the cryogenic receptacle to the heat exchanger, through these openings. Preferably, the cryogenic receptacle has a concave form with guiding ribs, reminiscent of a rib vault in architecture, for guiding the coolant through said openings.

Preferably, the cryogenic tip comprises a metal and/or a ceramic. This way, heat conduction from the skin lesion to the heat exchanger is enhanced, during the cryogenic treatment.

Preferably, the materials comprised by porous member have a low heat capacity. This way, these materials do not consume a significant part of the cooling capacity of the coolant, during the cryogenic treatment. For instance, the porous member comprises a foam material having a low heat capacity. Alternatively, the porous member comprises a sintered metal having a high thermal conductivity, combined with a high heat capacity. Preferably such sintered metal porous members are used in combination with metal tips, such that the heat conduction from the skin lesion is enhanced, during the cryogenic treatment.

According to a second, further preferred embodiment of the kit, alternative to the first further preferred embodiment above, the cryogenic applicator head comprises two or more ribs that radially surround a volume, whereby said volume is configured for receiving a cryogenic tip; in the engaged configuration of the cryogenic body, the cryogenic tip is in coolant communication with the cryogenic receptacle. Preferably, said ribs have straight inner edges, parallel to the axial direction. Said edges radially encapsulate the lateral sides of said volume at certain angular positions and/or angular intervals. The volume thus has a half-open lateral structure, whereby the lateral openings are breached by the inner sides of the ribs. The volume also has an axial opening, pointing away from the cryogenic applicator head. A cryogenic tip is provided in said volume via said opening.

In the engaged configuration of the cryogenic body, said cryogenic tip directly receives the coolant when the valve of the coolant container is opened. Thereafter, the coolant cools the cryogenic tip via evaporative cooling. A first option for the cryogenic treatment is to bring the tip immediately in contact with the skin lesion. The remainder of the coolant then directly cools said skin lesion. An alternative option is to wait until most of the coolant has evaporated. The fully cooled tip is subsequently brought in contact with the skin lesion. In the cryogenic treatment according to this second option, there advantageously is no direct contact between the coolant and the skin lesion.

An advantage of such a half-open lateral structure, is that the transfer of coolant from the cryogenic receptacle to the cryogenic tip is greatly enhanced.

An advantage of such a half-open lateral structure, is that the evaporation of the coolant is greatly enhanced: the gas thereby formed can now also escape towards the lateral sides.

Preferably, the cryogenic applicator head comprises three or more ribs, more preferably more than three ribs and less than 100 ribs, more preferably less than 24 ribs. More preferably, the cryogenic applicator head comprises 6, 7, 8, 9, 10, 11, or 12 ribs. In determining the number of ribs he/she should provide, the person skilled in the art balances, amongst other things, the enhanced evaporation in a structure having less ribs and the enhanced encapsulation of the cryogenic tip in a structure having more ribs.

In a further preferred embodiment of the kit, the kit comprises at least one, replaceable cryogenic tip. Preferably the kit comprises more than two but less than 50 replaceable cryogenic tips. Even more preferably, the number of cryogenic tips comprised by the kit roughly corresponds to the number of cryogenic treatments that can be performed with the coolant that is present in the coolant container. Even more preferably, the number of cryogenic tips comprised by the kit is slightly higher than said quantity. An advantage of employing replaceable tips is that it is more hygienic.

In a further preferred embodiment of the kit, said cryogenic tip is a foam tip. In case more than one tip is comprised, each of said tips is a foam tip. Preferably, a polyester-polyurethane foam is employed. More preferably, said foam has a cell diameter between 450 μm and 650 μm.

An advantage of such foam tips is that they are relatively cheap. Another advantage of employing said foam material is that it has a low heat capacity. Another advantage of employing foam tips having said pore size is that they sufficiently absorb the coolant, and let it evaporate in a gradual way such that the cold temperature is maintained for a longer period of time.

In a further preferred embodiment of the kit, the length of the kit is, in its engaged configuration, between 60 mm and 240 mm. More preferably, said length is greater than 80 mm. More preferably, said length is greater than 100 mm. More preferably, said length is less than 220 mm. More preferably, said length is less than 200 mm. More preferably, said length is between 160 mm and 170 mm, and even more preferably said length is about equal to 167 mm.

A kit having such a length in engaged configuration has several advantages. An advantage is that the kit fits in standard pharmacy storage racks and storage drawers, even/especially when standing upright. Another advantage is that such a kit is that it is feasible to make and allows an easy grip for “home-use”.

In a further embodiment, the coolant comprises dimethyl ether and/or propane. Preferably, the kit comprises a mixture of dimethyl ether (DME) and propane, called DMEP. Preferably, the DME content is between 0% and 100%, more preferably between 50% and 100%, and more preferably between 60% and 90%. Preferably, the DME content is between 0% and 100%, more preferably between 0% and 50% and more preferably between 10% and 40%. According to a non-limiting embodiment, the DME content is about equal to 75% and the propane content is about equal to 25%.

In another embodiment, the coolant comprises isopentane, propane, and/or 1,1-difluoroethane (which is also known as HFC-152a). Preferably, the kit comprises a isopentane, propane, and 1,1-difluoroethane. Optionally, the kit further comprises isobutane. Preferably, the joint content of 1,1-difluoroethane and isobutane (if present) is between 0% and 100%, more preferably between 50% and 100%. An advantage of employing such coolants and/or coolant mixtures, rather than liquid nitrogen, is that it is easier to store and to handle. Another advantage of employing such a coolant is that it is sufficiently capable of cooling the root region of the wart. This is at least the case when used in combination with cryogenic applicator heads of the above-described types, on the condition that their design has been optimized.

In a further preferred embodiment, the acidic composition comprises salicylic acid, formic acid, lactic acid, trichloroacetic acid, monochloroacetic acid, any fruit acid, and/or any combination of these acids. It is preferred that the acidic composition at least comprises trichloroacetic acid (TCA). It is further preferred that the TCA concentration in the acidic composition is between 20% and 60%. More preferably, said concentration is about 40%. TCA advantageously has a keratolytic action and is both safe for use in and suitable for acid treatments of skin lesions such as warts. TCA also actively attacks the tissue of the wart. An advantage of TCA over salicylic acid, is that it has a better safety profile.

Preferably, the acidic composition is a gel-like substance. An advantage is that such gel-like substances are easy to apply to skin lesions, as they are less prone to flowing away from the region of first deposition. As a direct consequence, there is a reduced risk for the neighboring, healthy tissue to get in contact with the acidic composition.

Preferably, the acidic composition does not comprise hyaluronic acid. In an alternative embodiment, the acidic composition does comprise hyaluronic acid.

In a second aspect, the invention provides a dosage regimen suitable for the non-surgical treatment of skin lesions including warts, verrucae, corns, and skin tags; in particular, the dosage regimen is employed for treating a skin lesion, thereby comprising:
performing a cryogenic treatment to a skin lesion, and
applying 5 microliter up to 40 microliter of an acidic composition to said skin lesion, twice a day during two to eight days; said acidic composition comprises salicylic acid, formic acid, lactic acid, trichloroacetic acid, monochloroacetic acid, one or more fruit acids, and/or any combination thereof.

Preferably, the quantity of the acidic composition that is applied to the wart thereby depends on the size of the wart.

Preferably, the “cryogenic treatment” is as introduced above, and entails a single treatment session in which the skin lesion is topically frozen. Preferably, the dosage regimen includes one or more of such cryogenic treatments. An “acid treatment”, as used hereunder and in the examples, relates to a single application of the acidic composition, whereby said acidic composition is the acidic composition as described above. Preferably, the dosage regimen includes more than one of such acid treatments.

An advantage of performing an acid treatment prior to a cryogenic treatment is that the acid treatment has, among other things, a keratolytic action. The acid treatment thereby weakens the tissue of the skin lesion. This makes it easier for the cryogenic treatment to act on the wart, thereby improving the efficacy of the cryogenic treatment.

An advantage of performing a cryogenic treatment prior to an acid treatment is that the cryogenic treatment has a necrotic effect. The cryogenic treatment thereby weakens the tissue of the skin lesion, such that the acidic composition can penetrate faster and/or deeper into the wart tissue. This improves the efficacy of the acid treatment.

Another advantage is that the combination of acid treatments and cryogenic treatments has a synergistic effect. It should be stressed that the cryogenic treatment and the acid treatment is not merely a pre-treatment and a treatment, in any order. The cryogenic treatment is a treatment on itself, and the acid treatment is a treatment on itself, since it includes at least one of the acids mentioned above.

Preferably, firstly a cryogenic treatment is performed, followed by several acid treatments. Optionally, a second and/or third cryogenic treatment is included. A synergistic effect is found when at least one of the cryogenic treatments is followed by one or more acid treatments, and for repetitive schemes following this order of treatment. A synergistic effect is also found when one or more acid treatments are followed by a cryogenic treatment, and for repetitive schemes following this order of treatment.

The order in which the cryogenic treatments and the acid treatments are performed can be inversed. Also, multiple cryogenic treatments can be included. Preferably, the dosage regimen is a (repetitive) scheme comprising one cryogenic treatment and multiple acid treatments. Either this scheme is executed once, or said scheme is executed two, three, or more than three times. Based on clinical tests, the man skilled in the art is able to determine how many repetitions are necessary for a significant percentage (50%, 60%, 70%, 80%, 90%, 95%, or 99%) of the skin lesions to have been fully treated. Alternatively, said scheme also comprises an evaluation as to whether the skin lesion has indeed been fully treated. If deemed necessary, in the light of said evaluation, the scheme is then repeated one, two, three, or more than three times, or it is repeated until the skin lesion has been fully treated. In the discussion above, the term “fully treated” denotes that the skin lesion has been removed. In case of warts, this means that it is very unlikely that the wart tissue will regrow. It is however possible that the skin tissue still needs healing.

Preferably, “twice a day”, or “two times a day” is once in the morning and once in the evening. Preferably, “three times a day” is once in the morning, once at about noon, and once in the evening.

A cryogenic treatment can be performed immediately after an acid treatment; for instance, about 5 minutes after the last acid treatment. Alternatively, the cryogenic treatment is performed on the day following to the last acid treatment. Preferably, an acid treatment is not performed immediately after a cryogenic treatment, but only the day after the last cryogenic treatment. This is because cryogenic treatments immediately followed by acid treatments can be painful and it can cause considerable damage to the tissue. Alternatively, the acid treatment is performed immediately after the last cryogenic treatment, for instance 5 minutes thereafter.

Prior to the first treatment, the area of the skin lesion to be treated is preferably soaked for 10 minutes in lukewarm water, and the skin lesion is filed with a pumice stone to remove any callus on the skin lesion. An advantage is that the tissue is then more susceptible to the treatment.

Preferably, the kit described above is employed in performing the treatment according to the dosage regimen described above.

In a preferred embodiment of the dosage regimen, a cryogenic treatment is performed on day 1. Starting from day two, an acid treatment is performed, twice a day during at least 1 and maximally 6 consecutive days. This is followed by a resting period of at least 1 and maximally 6 consecutive days, in which no acid treatments or cryogenic treatments are performed. Optionally on the last day, a second cryogenic treatment is performed.

The invention may thereto be described by the following embodiments:
1. A kit 1 suitable for the non-surgical treatment of skin lesions including warts, verrucae, corns, and skin tags, said kit comprising:
   a cryogenic body 2 comprising a housing 4 comprising a coolant container 29 and further comprising a cryogenic applicator head 5, whereby the housing 4 and the cryogenic applicator head 5 extend axially; said cryogenic applicator head 5 comprises an applicator end portion and is provided at said applicator end portion with a cryogenic tip 24 for performing a cryogenic treatment to a skin lesion, whereby said coolant container 29 comprises a coolant suitable for cooling said tip 24, and
   an acid body 3 comprising an acid container 6 comprising an acidic composition and having an acid outlet 10, and further comprising an acid dispenser head 7 that axially extends from said acid container 6; said acid dispenser head 7 is in fluid communication with said acid outlet 10 and is suitable for applying a dose of said acidic composition to a skin lesion.
2. The kit according to embodiment 1, characterized in that the cryogenic body 2 and acid body 3 are detachably engaged, thereby forming an elongated device.
3. The kit 1 according to embodiment 1, characterized in that the housing 4 and the cryogenic applicator head 5 are detachably engaged, thereby forming an engaged configuration.
4. The kit 1 according to any of embodiments 1 and 3, characterized in that the acid body 3 and the cryogenic body 2 are detachably engaged, thereby forming an engaged configuration.
5. The kit 1 according to previous embodiment 4, characterized in that the cryogenic applicator head 5 is positioned between the coolant container 29 and the acid body 3.
6. The kit 1 according to any of embodiments 4 and 5, characterized in that the cryogenic applicator head 5 comprises a distal end portion, opposite to its applicator end portion, whereby said distal end portion is configured for receiving the acid dispenser head 7.
7. The kit 1 according to any of embodiments 4 to 6, characterized in that the housing 4 comprises an end portion, whereby said end portion defines a cryogenic receptacle 30 that is configured for receiving the applicator end portion of the cryogenic applicator head 5.
8. The kit 1 according to previous embodiment 7, characterized in that the coolant container 29 has a coolant outlet 31 for the coolant and a coolant valve 32 communicating with said coolant outlet, whereby the cryogenic receptacle 30 is in coolant communication with said coolant outlet 31.
9. The kit 1 according to embodiment 8, characterized in that the cryogenic applicator head 5 comprises a heat exchanger that is in heat exchange contact with the cryogenic tip 24, said cryogenic tip 24 comprising a metal and/or a ceramic; in the engaged configuration of the cryogenic body 2, said heat exchanger is in coolant communication with the cryogenic receptacle 30; said heat exchanger further comprises a porous member adapted for conducting the coolant.
10. The kit 1 according to embodiment 8, characterized in that the cryogenic applicator head 5 comprises two or more ribs 26 that radially surround a volume, whereby said volume is configured for receiving a cryogenic tip 24; in the engaged configuration of the cryogenic body 2, said cryogenic tip 24 is in coolant communication with the cryogenic receptacle 30.
11. The kit 1 according to embodiment 10, characterized in that the kit 1 comprises at least one, replaceable cryogenic tip 24.
12. The kit 1 according to any of embodiments 10 and 11, characterized in that said cryogenic tip 24 is a foam tip.
13. The kit 1 according to any of embodiments 4 to 12, characterized in that the length of the kit 1 is between 60 mm and 240 mm.
14. The kit 1 according to any of the previous embodiments, characterized in that said coolant comprises isopentane, 1,1-difluoroethane, propane, isobutane, and/or dimethyl ether.
15. The kit 1 according to any of the previous embodiments, characterized in that said acidic composition comprises salicylic acid, formic acid, lactic acid, trichloroacetic acid, monochloroacetic acid, and/or one or more fruit acids.
16. A dosage regimen suitable for the non-surgical treatment of skin lesions including warts, verrucae, corns, and skin tags characterized in that the dosage regimen is employed for treating a skin lesion, thereby comprising the steps of:
   performing a cryogenic treatment to said skin lesion, and
   applying 5 microliter up to 40 microliter of an acidic composition to said skin lesion, twice a day during two to eight days; said acidic composition comprises salicylic acid, formic acid, lactic acid, trichloroacetic acid, monochloroacetic acid, one or more fruit acids, and/or any combination thereof.

### EXAMPLES

The invention is further described by the following non-limiting examples which further illustrate the invention, and are not intended to, nor should they be interpreted to, limit the scope of the invention.

The present invention will be now described in more details, referring to examples that are not limitative.

In a first embodiment of the dosage regimen, an acid treatment is performed, twice a day during four consecutive days (day 1 to day 4). On the last day (day 4), a cryogenic treatment is performed, about 5 minutes after the second acid treatment. This is followed by a resting period of four days (day 5 to day 8), in which no acid or cryogenic treatments are performed. Either this scheme is not repeated, or it is repeated one, two, or three times. Alternatively, the scheme comprises an evaluation on day 8 or on day 9, as to whether the skin lesion has been fully treated. If deemed necessary, in the light of said evaluation, the scheme is then repeated one, two, or three times, or it is repeated until the skin lesion is fully treated.

In a second embodiment of the dosage regimen, a cryogenic treatment is performed on day 1. Starting from day two, an acid treatment is performed, twice a day during four consecutive days (day 2 to day 5). This is followed by a resting period of four days (day 6 to day 9), in which no acid treatments or cryogenic treatments are performed. Optionally on day 10, a second cryogenic treatment is performed.

In a third embodiment of the dosage regimen, a cryogenic treatment is performed on day 1. Starting from day two, an acid treatment is performed, twice a day during four consecutive days (day 2 to day 5). This is followed by a resting period of four days (day 6 to day 9), in which no acid treatments or cryogenic treatments are performed. Either this scheme not repeated, or it is repeated one, two, or three times. Alternatively, the scheme comprises an evaluation on day 9 or on day 10, as to whether the skin lesion has been fully treated. If deemed necessary, in the light of said evaluation, the scheme is then repeated one, two, or three times, or it is repeated until the skin lesion is fully treated.

In a first embodiment, the coolant may comprise about 3% isopentane, about 88% 1,1-difluoroethane, and about 9% propane. In a second embodiment, the coolant may comprise about 5% isopentane, about 30% isobutane, about 30% 1,1-difluoroethane, and about 35% propane. Both coolant compositions may apply to any of the above dosage regimens.

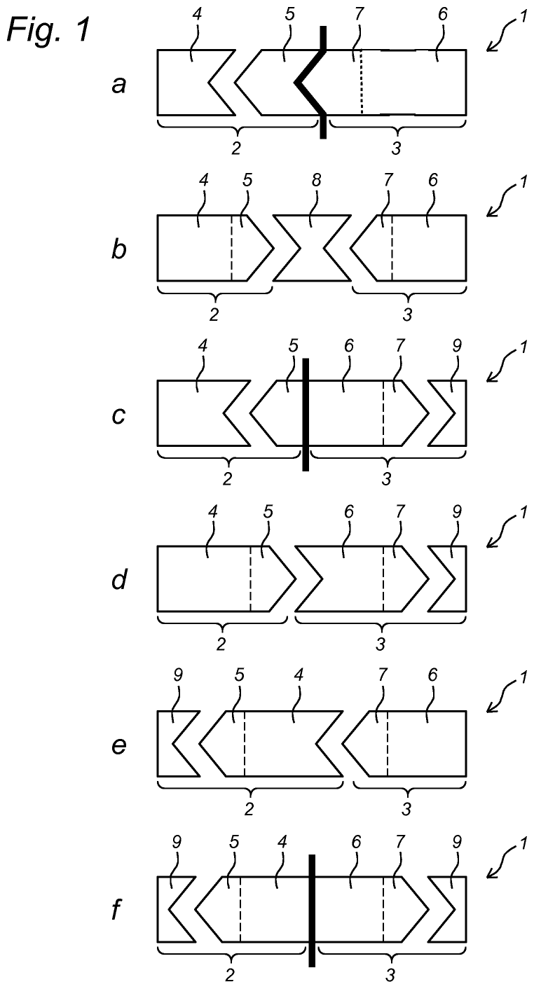

FIG. 1 schematically depicts a non-exhaustive collection of possible embodiments of the kit 1 according to the present invention. The kit 1 comprises both a cryogenic body 2 and an acid body 3.

Each of the kits (according to the different embodiments) comprises two or more parts that can be detachably engaged, thereby forming an engaged configuration of the kit 1. In the present, schematic pictures, such engagements are represented by parts having opposing, angular end portions that are convex and concave respectively. Said end portions thereby schematically fit together. In the particular case of the cryogenic applicator head 5, its applicator end portion corresponds with the convex triangular end portion. All the embodiments of FIG. 1 are designed in such a way that the engaged configuration shields both the cryogenic applicator head 5 and the acid dispenser head 7.

FIG. 1a depicts a preferred embodiment of the kit 1. The kit 1 thereby comprises a cryogenic body 2 and an acid body 3, that are detachably engaged at the thick line on the figure. The cryogenic body 2 comprises a housing 4 hosting the coolant container and a cryogenic applicator head 5. The cryogenic applicator head 5 can be detachably engaged with the housing 4 via its applicator end side. In a similar fashion, coolant is supplied from the coolant container to the cryogenic applicator head. The acid body 3 comprises an acid container 6 and an acid dispenser head 7. The kit does not comprise connection pieces 8. Nor does it comprise end caps 9. This is advantageous, because typically such small pieces easily get lost. Another advantage of the present embodiment is that the acid body 3 has a free end portion, opposite to its acid dispenser head 7. It is thus possible to provide some kind of control at this free end portion, for controlling the supply of the acidic composition.

FIG. 1b depicts an alternative embodiment of the kit 1. In particular, the cryogenic applicator head 5 is connected to the housing 4 hosting the coolant container. In such configurations, the cryogenic applicator head 5 is supplied with coolant via its end portion opposite to its applicator end portion. The kit 1 according to this embodiment comprises a connection piece 8.

FIG. 1c depicts yet an alternative embodiment of the kit 1. The cryogenic applicator head 5 is now directly connected to or detachably engaged with the acid container 6 via its distal end portion, at the thick line on the figure. The kit 1 according to this embodiment comprises an end cap 9.

FIG. 1d depicts yet an alternative embodiment of the kit 1. The applicator end portion of the cryogenic applicator head 5 can now be detachably engaged with an end portion of the acid container 6. The kit 1 according to this embodiment comprises an end cap 9.

FIG. 1e depicts yet an alternative embodiment of the kit 1. The acid dispenser head 7 can now be detachably engaged with an end portion of the housing 4 hosting the coolant container. The kit 1 according to this embodiment comprises an end cap 9.

FIG. 1f depicts yet an alternative embodiment of the kit 1. The housing 4 hosting the coolant container is now directly connected to or detachable engaged with the acid container 6, at the thick line on the figure. The kit 1 according to this embodiment comprises two end caps 9.

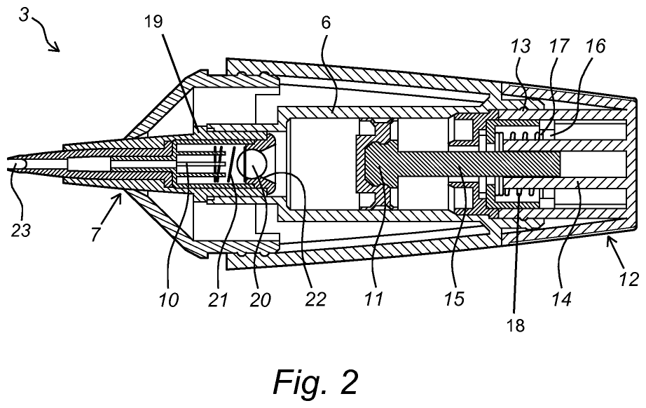

FIG. 2 shows an axial cut of the acid body 3, according to an embodiment of the invention. The acid body 3 is an elongated part, comprising an acid dispenser head 7 and an acid container 6 containing the acidic composition and having an acid outlet 10 towards said acid dispenser head 7. The acid container 6 is an elongated cylinder that is internally provided with an axial piston 11. Said piston 11 can be operated from the outside, via a rotary control 12 that is located at a first end portion of the acid body 3. The acid dispenser head 7 is provided at a second end portion of the acid body 3, opposite to the first one. The rotary control 12 is connected to the first end portion of the acid body via a click system that involves a transverse rib-in-groove connection 13. Under normal use, said rotary control 12 is therefore only capable of rotating with respect to the remainder of the acid body 3: the rib-in-groove 13 clicking system makes sure that both parts cannot be separated axially under normal use. The piston 11 is connected to a piston rod 15. The rotary control 12 comprises an internal bus 14, preferably a keyed bus in which the piston rod 15 can slide freely in the axial direction, yet whereby the rotary control 12 transfers its rotation to said piston rod 15. On the other hand, the piston rod 15 is provided with external thread, co-acting with the internal thread of a part that closes the first end of the acid container 6. The rotary control 12, the piston 11 and the piston rod 15 are arranged in such a way that the rotary control 12 operates the piston 11. Furthermore, the rotary control 12 is a one-way rotary control, whereby the only admissible turning direction corresponds to the piston 11 advancing into the acid container 6. To this end, the rotary control 12 comprises a first saw toothed ring 16, co-acting with an opposing, second saw toothed ring 17. The latter ring is pressed against the former, by means of a positioning spring 18. Contrary to the former ring, the latter ring is deterred from rotation, for example via longitudinal ribs, co-acting with longitudinal grooves. The nature of the saw toothed profiles is additionally such that only one relative rotation direction of both rings 16/17 is admissible. Said rotation direction corresponds to the piston 11 advancing into the acid container 6, as mentioned above. In the retracted position of the piston 11, the available volume in the acid container 6 is 1.5 ml, in a first embodiment. In alternative embodiment, this volume is 4.0 ml. In fact the volume needed can be readily estimated by the man skilled in the art. Preferably, the major share of this volume is taken by the acidic composition. The internal volume of the acid container 6 communicates with the outlet 10 of the acid container 6 via a ball-type relief valve 19. Said ball-type relief valve 19 comprises a ball 20 and a compensation spring 21, whereby the compensation spring 21 compensates for the hydrostatic pressure that is exerted on the ball 20 by the acidic composition. In doing so, the compensation spring 21 holds the ball 20 in an outwardly pointing, conical opening 22. Upon actuation of the rotary control 12, the piston 11 advances into the acid container 6. This causes a build-up of hydrostatic pressure in the acidic composition, up to the point where the compensation spring 21 is partly compressed. The compensation spring 21 now no longer holds the ball 20 in the conical opening 22; the ball 20 leaves the conical opening 22 due to the elevated hydrostatic pressure. At this point, some of the acidic composition escapes towards the acid outlet 10 until the hydrostatic pressure in the acidic composition is normalized, causing the ball-type relief valve 19 to close again. Advantageously, this dispensing system allows for an easy-controllable release of acidic composition. Of course, other dispensing systems can be readily implemented, as will be affirmed by the person skilled in the art. The acid outlet 10 of the acid container 6 communicates with the acid dispenser head 7. Preferably, the acid dispenser head 7 comprises an elongated, hollow structure with a small acid dispenser opening 23 at its distal end. Said opening 23 has a diameter no bigger than 2 mm, preferably no bigger than 1 mm. Such dispensing heads allow for a precise application of the acidic composition.

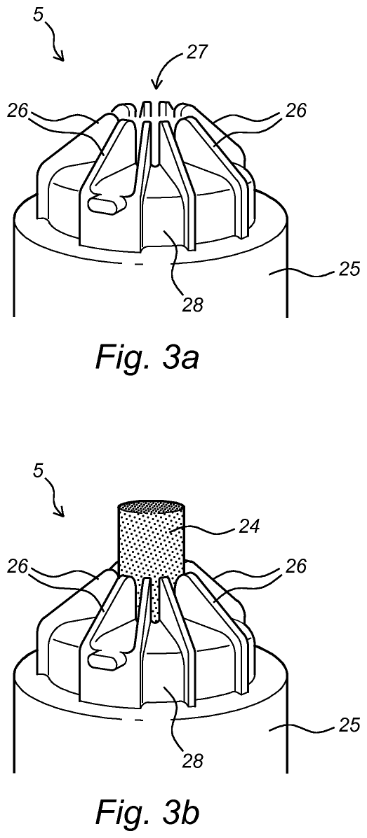

FIGS. 3a and 3b give a perspective view on the applicator end portion of the cryogenic applicator head 5 according to an embodiment of the invention, respectively without and with a cryogenic tip 24. The cryogenic applicator head 5 holds a cryogenic tip 24 or is configured for holding such a tip 24. Preferably, the cryogenic applicator head 5 is made of polypropylene, such that it may accidently contact the TCA acidic composition. In the present embodiment, the cryogenic applicator head 5 comprises a base portion 25 and nine ribs 26. In alternative embodiments, the cryogenic applicator head comprises a different number of ribs 26, for instance eight ribs. Said base portion 25 has a substantially flat or cone-like end, and said nine ribs 26 are provided on this flat end, perpendicularly thereto. Furthermore, said nine ribs 26 radially surround a cylindrical volume that perpendicularly extends from said flat end. This cylindrical volume has a circular cross section. The ribs 26 are well-distributed along the circumference the cross sectional circle; preferably, the ribs 26 are located at regular angular intervals. As such, said cylindrical volume defines a cylindrical tip receptacle 27, having a lateral structure that is partially open, and having a circular opening at its distal end, said opening pointing away from the base portion 25. At regular angular intervals, its laterally open structure is breached by the ribs 25. Such a tip receptacle 27 is suitable for receiving a cryogenic tip 24 that has a cylindrical base, for instance a substantially cylindrical tip 24. By preference, the tip 24 is a porous tip 24, comprising a foam material and/or a sintered metal. The fact that the tip receptacle 27 has a laterally open structure enhances evaporation of coolants absorbed by such tips 24. For instance, other suitable tip 24 forms have a substantially cylindrical base, the distal end portion of which is pointed or dome-shaped. Preferably, the ribs 26 are thin, slat-shaped structures that are positioned radially, with an inner edge pointing towards the thus defined tip receptacle 27, and having an outer edge pointing away from said receptacle 27. Even more preferably, the inner edge is substantially straight; these edges then extend perpendicularly from the flat end of the base portion 25. It is additionally preferred that the distal end portion of said inner edges is rounded off, such that a cryogenic tip 24 can be inserted more easily. Of course, it is equally possible for the ribs 26 to define tip receptacles 27 having other cross-sections. For instance, suitable tip receptacles 27 have a triangular, circular, square, or any other, regular or irregular cross section. Said tip receptacles 27 are then suitable for receiving cryogenic tips 24 having the corresponding cross section. The inner edges of the ribs 26 optionally form inwardly pointing lips, co-acting with recesses that are provided in the cryogenic tips 24, for reasons of improved grip. For instance, the inner edges of the ribs 26 then act as lips. Also, the number of ribs 26 can be different than nine. Preferably, the number of ribs 26 is more than one. For example, a sufficiently rigid tip 24 is used in combination with only two ribs 26. Said ribs 26 are provided at opposite sides and preferably co-act with grooves that are provided along the side of the tip 24. Alternatively, suitable cryogenic tips 24 comprise three ribs 26, four ribs 26, five ribs 26, six ribs 26, seven ribs 26, eight ribs 26, nine ribs 26, ten ribs 26, eleven ribs 26, twelve ribs 26, thirteen ribs 26, fourteen ribs 26, fifteen ribs 26, sixteen ribs 26, seventeen ribs 26, eighteen ribs 26, nineteen ribs 26, twenty ribs 26, twenty-one ribs 26, twenty-two ribs 26, twenty-three ribs 26, or twenty-four ribs 26. A very fine distribution of very fine ribs 26 (up to 60 or even up to 100 ribs 26) can be advantageous; the cold temperature of the tip 24, upon supply of the coolant, is then maintained for a longer period of time because the structure is less open. Optionally, the ribs 26 are transversally connected to each other near the base portion 25, as is the case on the figure. Preferably the base portion 25, the ribs 26, and the transverse rib connection 28, if present, form one integral part. Preferably, the distal end portion of the cryogenic applicator head 5 is configured for receiving and detachably engaging the acid dispenser head 7.

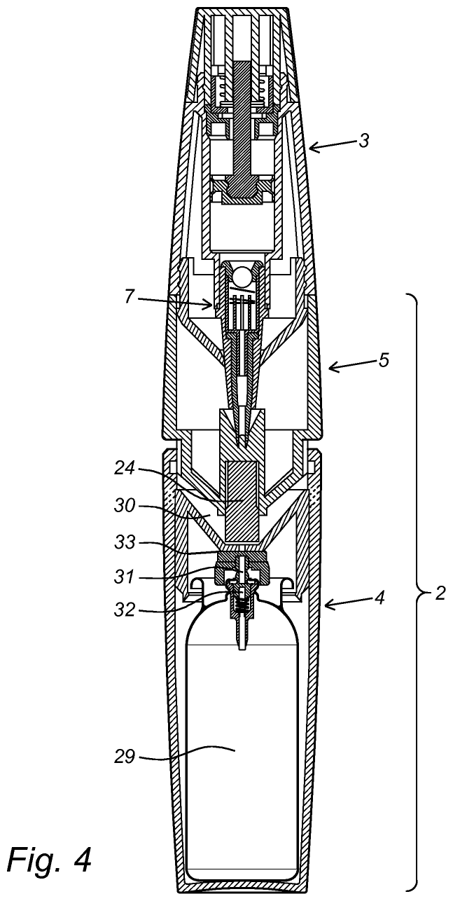

FIG. 4 shows an axial cut of the kit 1 in its engaged configuration, according to an embodiment of the invention. The kit 1 comprises a cryogenic body 2 and an acid body 3, whereby said cryogenic body 2 comprises a housing 4 hosting a coolant container 29, and further comprises a cryogenic applicator head 5. Said housing 4 has an end portion that forms a cryogenic receptacle 30. Said cryogenic receptacle receives and detachably engages the applicator end portion of the cryogenic applicator head 5; said head is provided with a cryogenic tip 24. The distal end portion of the cryogenic applicator head 5 receives and detachably engages the acid dispenser head 7 of the acid body 3. Said acid body 3 corresponds to its embodiment of FIG. 2. Said cryogenic applicator head 5 has an applicator end portion according to the embodiment of FIGS. 3a and 3b. The coolant container 29 has a coolant outlet 31 for the coolant and a coolant valve 32 communicating with said outlet 31. In the present embodiment, the coolant container is a standard gas cartridge having a standard outlet that opens up when exerting a sufficiently high pushing force. Each pair of detachably engaged parts of the kit can only be separated from each other when the relative azimuthal angle about their axis takes an exact value out of one or more specific, discrete values. The parts are then separated by exerting a relative pulling force. In the specific case of the cryogenic body 2, it is also possible to exert a pushing force on the cryogenic applicator head 5 relative to the housing 4 hosting the coolant container. If their relative azimuthal angle takes a correct value, the cryogenic applicator head 5 then advances into the housing, and pushes (e.g. via the cryogenic tip 24) against a pusher transmission 33. This causes said pusher transmission 33 to further advance into the housing 4. The pusher transmission 33 thereby pushes against the coolant valve 32. For a sufficiently high pushing force, the valve 32 is opened and the coolant is released. The coolant thereby enters the cryogenic receptacle 30 and cools the cryogenic tip 24 via evaporative cooling. The design ensures that it is impossible or at least very difficult for the user to open the valve directly using his/her fingers.

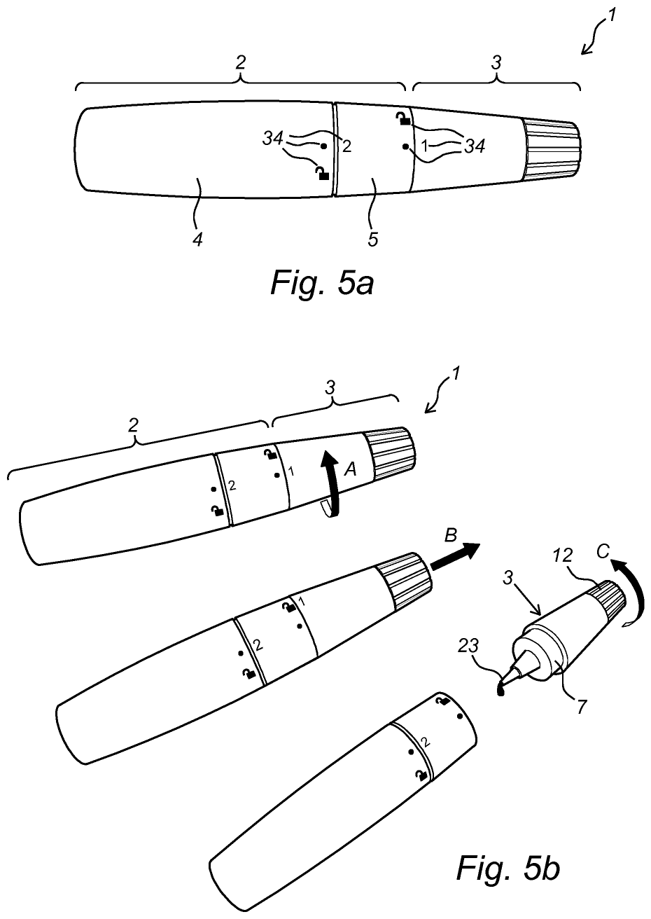

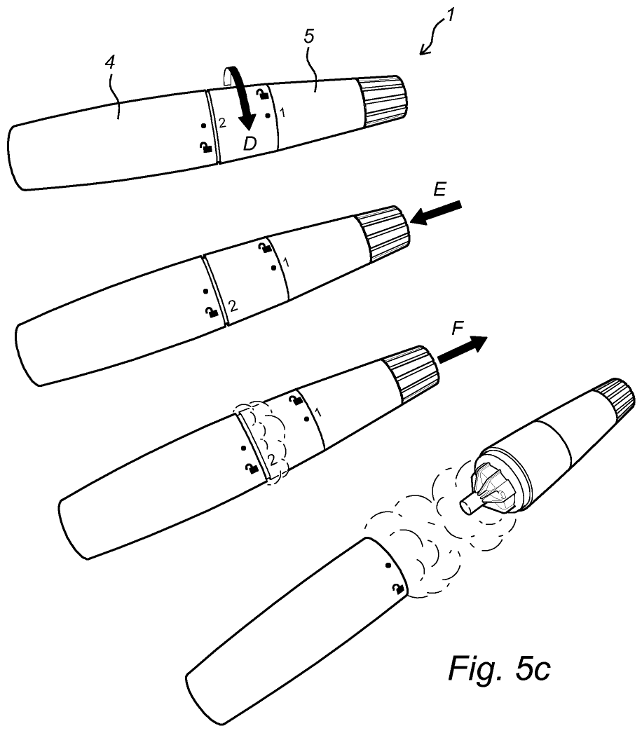

FIG. 5a gives a perspective view on the kit 1 in its engaged configuration, according to an embodiment of the invention. The kit 1 comprises a cryogenic body 2 and an acid body 3. The cryogenic body 2 comprises a housing 4 hosting a coolant container; it further comprises a cryogenic applicator head 5. The acid body comprises a rotary control 12 at its distal end. The visible sides of the housing 4, of the cryogenic applicator head 5, and of the acid body 3 are provided with icons 34. These icons give indications on how to separate the detachably engaged parts, on how to detachably engage the separated parts, and in which order the parts of the kit should be used.

FIG. 5b gives a perspective view on the kit 1, during steps prior to an acid treatment. In step A, the correct azimuthal angle of the acid body 3 relative to the cryogenic body 2 is installed. To this end, the acid body 3 is rotated about its axis, in such a way that the icon 34 visualizing a “1” is brought at the level of the icon 34 visualizing an unlocked lock. In step B, the acid body 3 is pulled out of the cryogenic body 2. In step C, the rotary control 12 is operated, such that the acidic composition is dispensed through the dispenser opening 23 of the acid dispenser head 7.

For performing an actual acid treatment, the acid body is held vertically with the dispenser head facing downwards. The rotary control is slowly turned clockwise to dispense the acidic composition. This way, one drop is carefully applied to cover the tissue of the skin lesion. Contact of the acidic composition with healthy skin is avoided as much as possible. The acidic composition is then left to dry for 10 to 15 minutes.

FIG. 5c gives a perspective view on the kit 1, during steps prior to a cryogenic treatment. In step D, the correct azimuthal angle of the cryogenic applicator head 5 relative to the housing 4 is installed. To this end, the cryogenic applicator head 5 is rotated about its axis, in such a way that the icon 34 visualizing a “2” is brought at the level of the icon 34 visualizing an unlocked lock. In step E, the cryogenic applicator head 5 is pushed into the housing 4, such that the coolant is released to the cryogenic tip 24. In step F, the cryogenic applicator head 5 (having a cooled tip) is pulled out of the housing 4.

For performing an actual cryogenic treatment, a foam tip is placed in the foam holder. The housing is held firmly with one hand and the cryogenic applicator head, detachably engaged with the acid body, is pushed into the housing until a hissing sound is heard. This pushing is maintained for about one second. In a next step, the cryogenic applicator head is pulled out of the housing. The foam tip is saturated with coolant. It is exposed to the air during 20 seconds, for said coolant to evaporate partly. Then, the cryogenic tip is pressed on the tissue of the skin lesion during about 20 to about 40 seconds, depending on the type of skin lesion: 20 seconds for hand warts, 40 seconds for foot warts. The cryogenic tip is disposed of, after having waited for another two minutes.

As can be seen in FIG. 5, both detachable engagements are released by turning in a counterclockwise direction, when looking in the direction of the cryogenic tip and the acid dispensing head. The icons indicating “1” and “2”, indicate in which order the treatments should be applied. Of course, said order can be turned around. In alternative embodiments, detachably engaged parts can be rotated freely with respect to each other, 360° in both directions about the axis. The icons thereby indicate the specific relative rotation angles of interest.

It is supposed that the present invention is not restricted to any form of realization described previously and that some modifications can be added to the presented example of fabrication without reappraisal of the appended claims.

### FEATURES RELATING TO THE FIGURES

1. kit
2. cryogenic body
3. acid body
4. housing
5. cryogenic applicator head
6. acid container
7. acid dispenser head
8. connection piece
9. end cap
10. acid outlet
11. piston
12. rotary control
13. rib-in-groove connection
14. internal bus
15. piston rod
16. first saw toothed ring
17. second saw toothed ring
18. positioning spring
19. ball-type relief valve
20. ball
21. compensation spring
22. conical opening
23. dispenser opening
24. cryogenic tip
25. base portion
26. rib
27. tip receptacle
28. transverse rib connection
29. coolant container
30. cryogenic receptacle
31. coolant outlet
32. coolant valve
33. pusher transmission
34. icon

1. A kit (1) suitable for the non-surgical treatment of skin lesions including warts, verrucae, corns, and skin tags, said kit comprising:
a cryogenic body (2) comprising a housing (4) comprising a coolant container (29) and further comprising a cryogenic applicator head (51, whereby the housing (4) and the cryogenic applicator head (51 extend axially; said cryogenic applicator head (51 comprises an applicator end portion and is provided at said applicator end portion with a cryogenic tip (24) for performing a cryogenic treatment to a skin lesion, whereby said coolant container (29) comprises a coolant suitable for cooling said tip (24), and
an acid body (3) comprising an acid container (6) comprising an acidic composition and having an acid outlet (10), and further comprising an acid dispenser head (7) that axially extends from said acid container (6); said acid dispenser head (7) is in fluid communication with said acid outlet (10) and is suitable for applying a dose of said acidic composition to a skin lesion.

2. The kit according to claim 1, characterized in that the cryogenic body (2) and acid body (3) are detachably engaged, thereby forming an elongated device.

3. The kit (1) according to claim 1, characterized in that the housing (4) and the cryogenic applicator head (5) are detachably engaged, thereby forming an engaged configuration.

4. The kit (1) according to claim 1, characterized in that the acid body (3) and the cryogenic body (2) are detachably engaged, thereby forming an engaged configuration.

5. The kit (1) according to claim 4, characterized in that the cryogenic applicator head (5) is positioned between the coolant container (29) and the acid body (31.

6. The kit (1) according to claim 4, characterized in that the cryogenic applicator head (5) comprises a distal end portion, opposite to its applicator end portion, whereby said distal end portion is configured for receiving the acid dispenser head (7).

7. The kit (1) according to claim 4, characterized in that the housing (4) comprises an end portion, whereby said end portion defines a cryogenic receptacle (30) that is configured for receiving the applicator end portion of the cryogenic applicator head (5).

8. The kit (1) according to claim 7, characterized in that the coolant container (29) has a coolant outlet (31) for the coolant and a coolant valve (32) communicating with said coolant outlet, whereby the cryogenic receptacle (30) is in coolant communication with said coolant outlet (31).

9. The kit (1) according to claim 8, characterized in that the cryogenic applicator head (5) comprises a heat exchanger that is in heat exchange contact with the cryogenic tip (24), said cryogenic tip (24) comprising a metal and/or a ceramic; in the engaged configuration of the cryogenic body (2), said heat exchanger is in coolant communication with the cryogenic receptacle (30); said heat exchanger further comprises a porous member adapted for conducting the coolant.

10. The kit (1) according to claim 8, characterized in that the cryogenic applicator head (5) comprises two or more ribs (26) that radially surround a volume, whereby said volume is configured for receiving a cryogenic tip (24); in the engaged configuration of the cryogenic body (21, said cryogenic tip (24) is in coolant communication with the cryogenic receptacle (30).

11. The kit (1) according to claim 10, characterized in that the kit (1) comprises at least one, replaceable cryogenic tip (24).

12. The kit (1) according to claim 10, characterized in that said cryogenic tip (24) is a foam tip.

13. The kit (1) according to claim 4, characterized in that the length of the kit (1) is between 60 mm and 240 mm.

14. The kit (1) according to claim 1, characterized in that said coolant comprises isopentane, 1,1-difluoroethane, propane, isobutane, and/or dimethyl ether.

15. The kit (1) according to claim 1, characterized in that said acidic composition comprises salicylic acid, formic acid, lactic acid, trichloroacetic acid, monochloroacetic acid, and/or one or more fruit acids.

16. A dosage regimen suitable for the non-surgical treatment of skin lesions including warts, verrucae, corns, and skin tags characterized in that the dosage regimen is employed for treating a skin lesion, thereby comprising the steps of:
performing a cryogenic treatment to said skin lesion, and
applying 5 microliter up to 40 microliter of an acidic composition to said skin lesion, twice a day for two to eight days; said acidic composition comprises salicylic acid, formic acid, lactic acid, trichloroacetic acid, monochloroacetic acid, one or more fruit acids, and/or any combination thereof.

17. The kit (1) according to claim 5, characterized in that the cryogenic applicator head (5) comprises a distal end portion, opposite to its applicator end portion, whereby said distal end portion is configured for receiving the acid dispenser head (7).

18. The kit (1) according to claim 5, characterized in that the housing (4) comprises an end portion, whereby said end portion defines a cryogenic receptacle (30) that is configured for receiving the applicator end portion of the cryogenic applicator head (5).

19. The kit (1) according to claim 6, characterized in that the housing (4) comprises an end portion, whereby said end portion defines a cryogenic receptacle (30) that is configured for receiving the applicator end portion of the cryogenic applicator head (5).
